(19) United States
(12) Patent Application Publication
(10) Pub. No.: US 2019/0290896 A1
(43) Pub. Date: Sep. 26, 2019

(54) TWO-STAGE LOCKING CATHETER SPLICE ASSEMBLY

(71) Applicant: Flowonix Medical Incorporated, Mt. Olive, NJ (US)

(72) Inventors: Arnold Phillips, Sharon, MA (US); Xiaonan Qin, Chester Springs, PA (US)

(21) Appl. No.: 15/934,232

(22) Filed: Mar. 23, 2018

(51) Int. Cl. A61M 39/12

(52) U.S. Cl. CPC A61M 39/12; A61M 2039/1027

(57) ABSTRACT

Various embodiments include a locking catheter splice assembly that may include a multi-stage barb fitting, a first locking member, and a second locking member. The multi-stage barb fitting may have a first end including a first set of barbs and a second end including a second set of barbs. The first end may be configured to be inserted into the first catheter and the second end may be configured to be inserted into the second catheter. The first locking member may be slipped over the first catheter so as to surround the portion of the first end of the multi-stage barb inserted into the first catheter. The second locking member may be slipped over the second catheter so as to surround the portion of the second end of the multi-stage barb inserted into the second catheter. The first and second locking members may interlock with one another when pressed together.

### BACKGROUND

Catheters are often assembled or extended by coupling two separate sections of catheter tubing. A fitting between the two separate catheter tubes is generally used to form a coupling. However, catheter outer diameters and inner diameters vary. As a result, many different sized fittings need to be stocked in a medical facility to be able couple together catheters of various sizes.

### SUMMARY

Various embodiments include a catheter coupling assembly configured to securely couple catheters of various sizes. In particular, various embodiments include a locking catheter splice assembly for joining a first catheter and a second catheter. The locking catheter splice assembly may include a multi-stage barb fitting, a first locking member, and a second locking member. The multi-stage barb fitting may have a first end including a first set of barbs and a second end including a second set of barbs. The first end may be configured to be inserted into the first catheter and the second end may be configured to be inserted into the second catheter. The first locking member may be configured to be slipped over the first catheter and to surround the portion of the first end of the multi-stage barb inserted into the first catheter. The second locking member may be configured to be slipped over the second catheter and to surround the portion of the second end of the multi-stage barb inserted into the second catheter. The first and second locking members may be configured to interlock with one another when pressed together.

In various embodiments, the first locking member may be configured to compress an outer diameter of a portion of the first catheter against at least one of the first set of barbs when the first and second locking members are interlocked with one another. The first locking member may include an inner cavity having a first catheter trapping surface separate from a second catheter trapping surface. The first and second catheter trapping surfaces may be axially aligned with a first barb and a second barb when the first locking member and the second locking member are interlocked with one another. Each of the first and second sets of barbs may include a first barb and a second barb, wherein the first barb has a smaller outermost diameter than the second barb. The multi-stage barb fitting may be symmetrical about the longitudinal axis. In various embodiments, one of the first and second locking members may include a latching tab configured to be received in a latching slot included in the other of the first and second locking members for interlocking the first and second locking members. The latching slot may extend radially through the other of the first and second locking members that includes the latching slot. The other of the first and second locking members that includes the latching slot may further include a guide slot configured to guide the movement of the first and second locking members toward one another along a longitudinal axis of the multi-stage barb fitting.

In various embodiments, the multi-stage barb fitting may include a central portion positioned between the first and second ends that is wider than the first and second sets of barbs. The central portion may include a gripping surface configured to facilitate gripping of the central portion during surgery. A gap may be formed between a largest outer diameter of the central portion and a largest inner diameter of at least one of the first and second locking members when the first and second locking members are mated together to couple the first and second catheters using the multi-stage barb.

Various embodiments include a method for joining a first catheter and a second catheter. The method may include slipping a first locking member over the first catheter. A second locking member may be slipped over the second catheter. A multi-stage barb fitting may be inserted into open ends of both the first and second catheters. A first end of the multi-stage barb fitting may include a first set of barbs and a second end of the multi-stage barb fitting may include a second set of barbs. In addition, the first and second locking members may be pressed together over the multi-stage barb fitting until the first and second locking members interlock.

### BRIEF DESCRIPTION OF THE DRAWINGS

The accompanying drawings, which are incorporated herein and constitute part of this specification, illustrate example embodiments, and together with the general description given above and the detailed description given below, serve to explain the features of the claims.

FIG. 1A is a cross-sectional view illustrating a locking catheter splice assembly, joining a first catheter and a second catheter at an initial stage of assembly in accordance with various embodiments.

FIG. 1B is a cross-sectional view illustrating the locking catheter splice assembly of FIG. 1A at a further stage of assembly in accordance with various embodiments.

FIG. 1C is a cross-sectional view illustrating the locking catheter splice assembly of FIGS. 1A and 1B at a final stage of assembly in accordance with various embodiments.

FIG. 2A is an isometric view illustrating the multi-stage barb fitting of FIGS. 1A-1C in isolation in accordance with various embodiments.

FIG. 2B is a front view illustrating the multi-stage barb fitting of FIG. 2A in isolation in accordance with various embodiments.

FIG. 2C is a longitudinal cross-section view illustrating the multi-stage barb fitting of FIGS. 2A and 2B in isolation in accordance with various embodiments.

FIG. 3A is an isometric view illustrating the first locking member of FIGS. 1A-1C in isolation in accordance with various embodiments.

FIG. 3B is a front view illustrating the first locking member of FIG. 3A in isolation in accordance with various embodiments.

FIG. 3C is a longitudinal cross-section view illustrating the first locking member of FIG. 3B at C-C therein in accordance with various embodiments.

FIG. 4A is an isometric view illustrating the second locking member of FIGS. 1A-1C in isolation in accordance with various embodiments.

FIG. 4B is a front view illustrating the second locking member of FIG. 4A in isolation in accordance with various embodiments.

FIG. 4C is a longitudinal cross-section view illustrating the second locking member of FIGS. 4A and 4B in isolation in accordance with various embodiments.

FIG. 5 is a longitudinal cross-sectional view illustrating the assembled locking catheter splice assembly of FIG. 1C in accordance with various embodiments.

FIG. 6 is an isometric view illustrating the assembled locking catheter splice assembly FIGS. 1C and 5 joining the first and second catheters in accordance with various embodiments.

FIG. 7 is a front view illustrating a translucent version the assembled locking catheter splice assembly in accordance with various embodiments.

FIG. 8 is an end view illustrating an alignment feature in first locking member in accordance with various embodiments.

FIG. 9 is a perspective view illustrating an alternative multi-stage barb fitting in accordance with various embodiments.

FIG. 10 is a process flow diagram illustrating a method of assembling a locking catheter splice assembly for joining a first catheter and a second catheter in accordance with various embodiments.

### DETAILED DESCRIPTION

The various embodiments will be described in detail with reference to the accompanying drawings. Wherever possible, the same reference numbers will be used throughout the drawings to refer to the same or like parts. References made to particular examples and implementations are for illustrative purposes, and are not intended to limit the scope of the claims.

Various embodiments include a locking catheter splice assembly for securely joining two catheters (referred to as a first catheter and a second catheter) which may be of different sizes. In various embodiments, the locking catheter splice assembly includes a multi-stage barb fitting, a first locking member and a second locking member. The multi-stage barb fitting is configured to have at least a portion of a first end thereof inserted into the first catheter and at least a portion of a second end thereof inserted into the second catheter. The first end of the multi-stage barb fitting may include a first set of barbs and the second end of the multi-stage barb fitting may include a second set of barbs. The first locking member may be configured to surround the portion of the first end of the multi-stage barb fitting with the first catheter inserted therein. Similarly, the second locking member may be configured to surround the portion of the second end of the multi-stage barb fitting with the second catheter inserted therein. The first and second locking members may include complementary locking structures configured to interlock with one another when the first and second locking members are mated together by moving the locking members toward one another along a longitudinal axis of the multi-stage barb fitting. The multi-stage barb fitting and the configurations of the first and second locking members enable connecting two different catheters together even if the two catheters having different inner diameters.

There are a number of medical devices that use catheters to administer a fluid, such as a medication, to a particular site within a patient. In particular, implantable drug delivery devices (also referred to herein as implantable infusion pumps) use catheters implanted within a patient for delivery of infusate, such as medication. Such implantable medical devices offer patients many benefits as compared to periodic injections, oral medication and medical devices located outside the patient. However, sometimes such medical devices need to be replaced.

Connections between two different catheters are often needed for revision or replacement surgeries involving an implanted medical device that uses a catheter (e.g., an implantable infusion pump). If an implanted medical device needs to be replaced for whatever reason, reusing the implanted catheter may result in less trauma to the patient. To reuse the implanted catheter, a clinician may need to splice part of a new catheter connected to a replacement medical device to that implanted catheter. This may require connecting two different types of catheters with two different diameters. The various embodiments facilitate such connections by providing a universal catheter splice coupling useful for joining two catheters that may have different inner diameters.

In use, a clinician may replace an implanted medical device, such as a drug delivery pump, by separating the medical device from an implanted catheter, and connecting a replacement medical device to the implanted catheter. For example, the clinician may cut the implanted catheter near its connection with the medical device, leaving a cut exposed end of the implanted catheter. The clinician may then connect a new catheter that is part of the replacement medical device to that cut exposed end of the implanted catheter using a locking catheter splice assembly in accordance with various embodiments. The clinician may determine an appropriate length of the new catheter that connected to the replacement medical device, and cut the new catheter to an appropriate length before joining the new catheter to the previously implanted catheter. This procedure enables the clinician to replace an implanted medical device, such as a drug delivery pump, without removing the previously implanted catheter, thereby reducing trauma to the patient from the procedure.

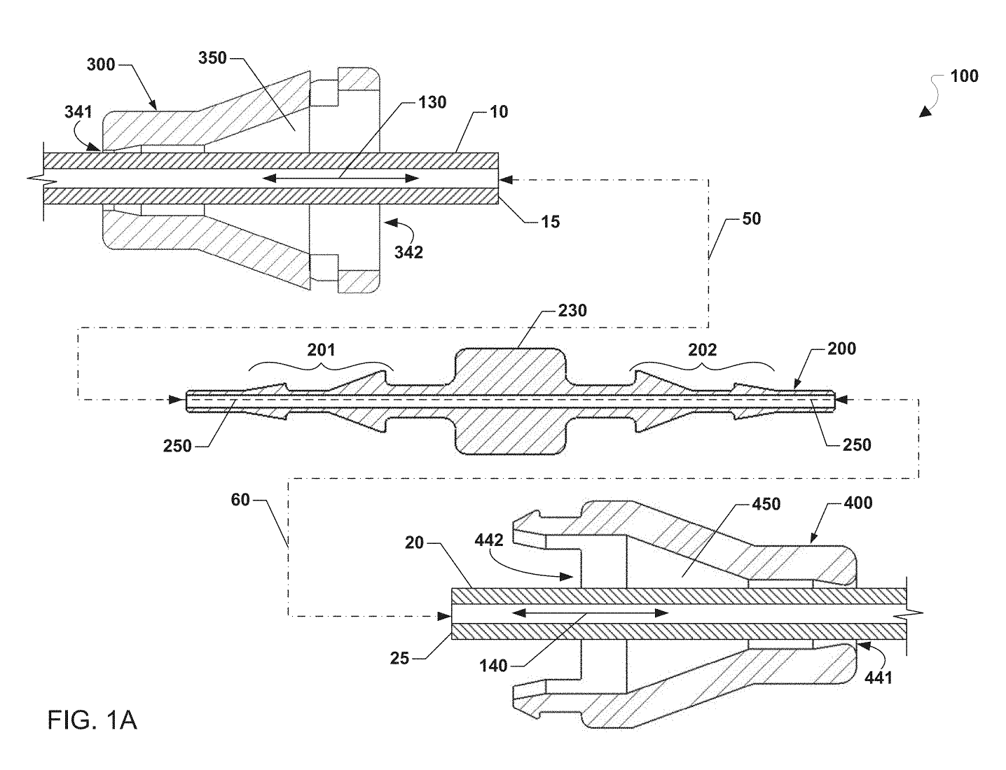

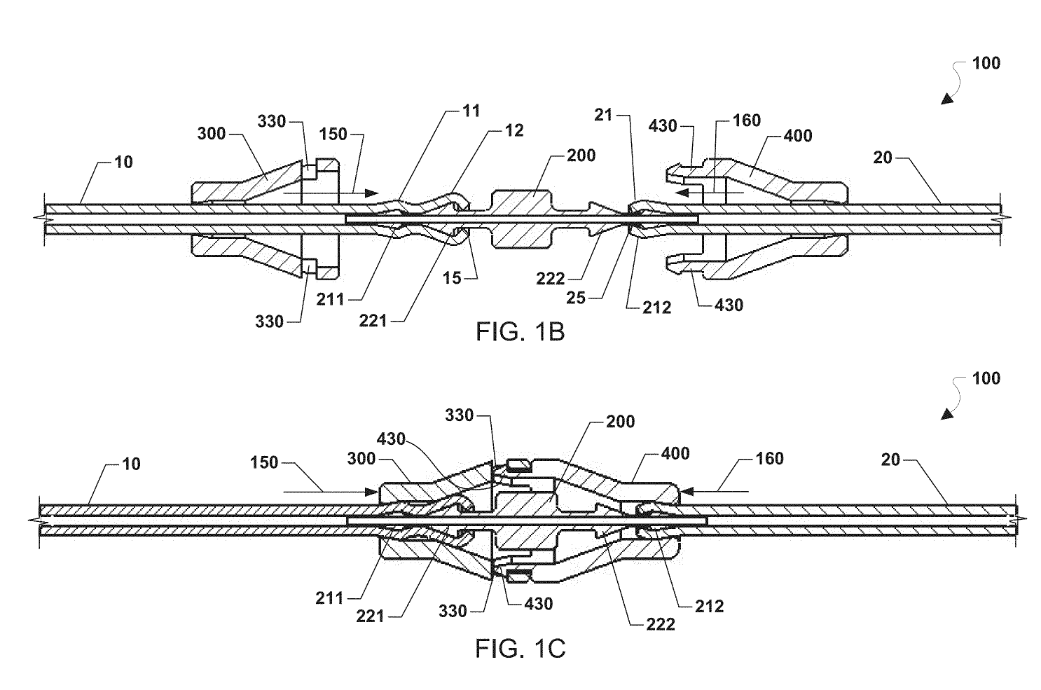

FIGS. 1A-1C illustrate an embodiment locking catheter splice assembly 100 for joining a first catheter 10 to a second catheter 20 at an initial stage of assembly. A remote end of one of the first and second catheters 10, 20 may be coupled to a medical device (not shown), such as an implantable infusion pump, and a remote end of the other one of the first and second catheters 10, 20 may be implanted in a patient. In contrast, the near ends 15, 25 of the first and second catheters 10, 20 are illustrated as being detached from anything and ready to receive elements of the locking catheter splice assembly 100. Prior to the first and second catheters 10, 20 being joined, a clinician may prepare the near ends 15, 25 of the first and second catheters 10, 20 so that the catheters are ready to be spliced together. For example, the clinician may cut off a segment from the ends of the first and second catheters 10, 20 to ensure the near ends 15, 25 have a clean cut.

FIG. 1A illustrates the locking catheter splice assembly 100 in an initial stage of assembly. As shown, the locking catheter splice assembly 100 may include a multi-stage barb fitting 200, a first locking member 300, and a second locking member 400. The multi-stage barb fitting 200 has a tubular shape with a central portion 230 and two ends, referred to herein as a first end (e.g., the left end in the orientation shown in FIG. 1A) and a second end (e.g., the right end in the orientation shown in FIG. 1A). For ease of describing the multi-stage barb fitting 200, the first end is described as fitting into the first catheter 10 (e.g., in the first open end 15) as indicated in dashed line 50, and the second end is described as fitting into the second catheter 20 (e.g., in the second open end 25 thereof) as indicated in dashed line 60. However, the multi-stage barb fitting 200 may be symmetrical such that either of the first and second ends may be inserted into either of the two catheters as described herein.

The multi-stage barb fitting 200 may include a first set of barbs 201 on a first end and a second set of barbs 202 on a second end. In the example embodiment illustrated in FIG. 1A, the first set of barbs 201 includes a first-end first barb 211 and a larger diameter first-end second barb 221, and the second set of barbs 201 includes a second-end first barb 212 and a larger diameter second-end second barb 222. While the figures illustrate two barbs on each end of the multi-stage barb fitting 200, more than two barbs may be included in some embodiments.

The first locking member 300 may be configured to be slipped over the first catheter 10 and then surround at least part of the first end of the multi-stage barb fitting 200 when the locking catheter splice assembly 100 finally assembled. Similarly, the second locking member 400 may be configured to be slipped over the second catheter 20 and then surround at least part of the second end of the multi-stage barb fitting 200 when the locking catheter splice assembly 100 finally assembled. The first and second locking members 300, 400 may be configured to interlock with one another when mated together by moving the first and second locking members 300, 400 toward one along the longitudinal axis 250 of the multi-stage barb fitting 200. In the example embodiment illustrated in FIGS. 1A-1C, the first locking member 300 includes two latching slots 330 that are each configured to receive a latching tab 430 axially projecting from the second locking member 400.

Before inserting the multi-stage barb fitting 200 into the first and second catheters 10, 20, the clinician slips the first and second locking members 300, 400 over of the first and second catheters 10, 20 as shown in FIG. 1A. The first locking member 300 may include outer walls forming a first inner cavity 350 between a first entry aperture 341 and a first egress aperture 342, opposite the first entry aperture 341. The first locking member 300 may be slipped onto the first catheter 10 by inserting the first open end 15 of the catheter into the first entry aperture 341, through the first inner cavity 350, and out the first egress aperture 342. Similarly, the second locking member 400 may include outer walls forming a second inner cavity 450 between a second entry aperture 441 and a second egress aperture 442, opposite the second entry aperture 441. The second locking member 400 may be slipped onto the second catheter 20 by inserting the second open end 25 of the catheter into the second entry aperture 441, through the second inner cavity 450, and out the second egress aperture 442. Once the first and second locking members 300, 400 are on the first and second catheters 10, 20, the locking members may be slid along the catheters to expose the ends 15, 25 for connecting to the multi-stage barb fitting 200.

FIG. 1B illustrates the locking catheter splice assembly 100 in an intermediate stage of assembly in which the multi-stage barb fitting 200 has been inserted into both the first and second catheters 10, 20. While the multi-stage barb fitting 200 is being inserted into the first and second catheters 10, 20, the first and second locking members 300, 400 may be slid distance away from the open ends 15, 25 of the first and second catheters 10, 20. With the first and second locking members 300, 400 out of the way, the clinician may push the open ends 15, 25 of the two catheters 10, 20 as far onto the multi-stage barb fitting 200 as the catheters will go. For example, the clinician may push the first catheter 10 in a first direction 150, relative to the multi-stage barb fitting 200, until the first open end 15 has slid over the first-end first barb 211 and, if the catheter is large enough, over the first-end second barb 221. Similarly, the clinician may push the second catheter 20 in a second direction 160, relative to the multi-stage barb fitting 200, until the first open end 25 has slid over the second-end first barb 212 and, if the catheter is large enough, over the second-end second barb 222.

In the example illustrated in FIGS. 1B and 1C, the first catheter 10 has a wider inner diameter and/or is made of a more flexible material than the second catheter 20, allowing the first catheter 10 to be slid further onto the multi-stage barb fitting 200 than the second catheter 20. Said another way, FIGS. 1B and 1C illustrate the multi-stage barb fitting 200 inserted further into the first catheter 10 than the second catheter 20. Once the multi-stage barb fitting 200 has been inserted as far as possible into the first catheter 10, a first-end first bulge 11 and a first-end second bulge 12 (i.e., when the catheter is covering the first-end second barb 221) may form in the first catheter 10 corresponding to the locations of the first-end first barb 211 and a first-end second barb 221, respectively. In contrast, when the second catheter 20 is pushed onto the multi-stage barb fitting 200, because the second catheter 20 has a smaller inner diameter and/or is less flexible, the second open end 25 may only be slid over a second-end first barb 212 and not a second-end second barb 222 of the multi-stage barb fitting 200. In this way, once the multi-stage barb fitting 200 has been inserted as far as possible into the second catheter 20, only one second-end bulge 21 may form in the second catheter 20 corresponding to the location of the second-end first barb 212. The second-end second barb 222 may remain uncovered by the second catheter 20.

FIG. 1C illustrates the locking catheter splice assembly 100 in a final stage of assembly in which the first and second locking members 300, 400 are interlocked and/or mated together, covering the multi-stage barb fitting 200 and the first and second open ends 15, 25 of the first and second catheters 10, 20. One of the first and second locking members 300, 400 may include a latching tab configured to be received in a latching slot included in the other of the first and second locking members 300, 400. As noted above, in the example embodiment illustrated in FIGS. 1A-1C, the first locking member 300 includes two latching slots 330 that are each configured to receive a separate latching tab 430 axially projecting from the second locking member 400. As an alternative, each the first and second locking members 300, 400 may include a latching tab and a latching slot, configured to mate with a corresponding latching slot and latching tab on the other of the first and second locking members 300, 400. Some embodiments may include just one or more than two latching tabs and corresponding latching slots. Other forms or configurations of interlocking members may also be used in some embodiments.

A clinician my interlock and/or mate together the first and second locking members 300, 400 by moving the first and second locking members 300, 400 toward one another, or at least one of the first and second locking members 300, 400 toward the other, until the latching tabs 430 snap into the latching slots 330. For example, the clinician may push the first locking member 300 in the first direction 150 while pushing the second locking member 400 in the second direction 160 until the latching tabs 430 snap into the corresponding latching slots 330. In some embodiments, the locking catheter splice assembly 100 includes a self-centering design that automatically centers the multi-stage barb fitting 200 between the first and second locking members 300, 400 when the two are mated together on the multi-stage barb fitting 200. For example, the center portion 230 of the multi-stage barb fitting 200 may have a diameter that matches with the inner diameter of the first and second locking members 300, 400.

As illustrated in FIG. 1C, the first and second locking members 300, 400 press against the bulges in the first and second catheters 10, 20 when the members are interlocked, compressing the catheters between surfaces on the locking members 300, 400 and one or more of the barbs 211, 212, 221, 222 of the multi-stage barb fitting 200. This compression holds the catheters 10, 20 on the multi-stage barb fitting 200 while the interlocking members 330, 430 holds the first and second locking members 300, 400 together, thus ensuring neither catheter will pull out of the locking catheter splice assembly 100.

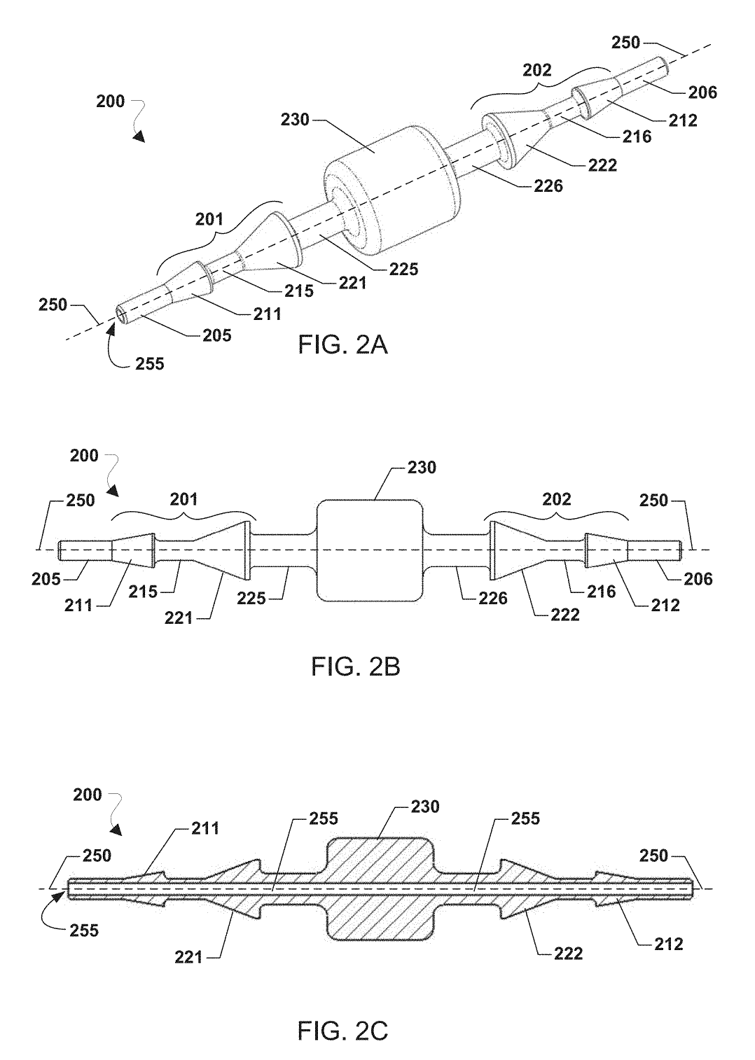

FIGS. 2A-2C illustrate a multi-stage barb fitting 200 in isolation in accordance with various embodiments. The multi-stage barb fitting 200 is configured to be inserted into two catheters with the same or different diameters to provide a splice connection between the two catheters. An inner lumen 255 may extend longitudinally through the full length of the multi-stage barb fitting 200 along a central longitudinal axis 250 thereof. As described above, the multi-stage barb fitting 200 may include a first set of barbs 201 on a first longitudinal end and a second set of barbs 202 on an opposite second longitudinal end. Each of the first and second sets of barbs 201, 202 is configured to receive a catheter. The first set of barbs 201 may include a first-end first barb 211 and a first-end second barb 221. Similarly, the second set of barbs 202 may include a second-end first barb 212 and a second-end second barb 222. The barbs 211, 212, 221, 222 may have a tapered conical outer surface that increases in outer diameter along the central longitudinal axis 250 in a direction toward a center of the multi-stage barb fitting 200. A largest diameter of the outer-most pair of barbs 211, 212 may be smaller than a largest diameter of the inner-most pair of barbs 221, 222, which may in-turn have a smaller largest diameter than a central fitting structure 230 of the multi-stage barb fitting 200. In addition, the multi-stage barb fitting 200 may include cylindrical landings 205, 206, 215, 216, 225, 226, which may have a constant or near constant outer diameter along the central longitudinal axis 250. The intermediate landings 215, 216 may provide an axial offset between each of the outer-most barbs 211, 212 and their adjacent inner-most barbs 221, 222. In addition, inner-most landings 225, 226 may be disposed between the inner-most barbs 221, 222 and the central fitting structures 230. The outermost landings 205, 206, which may be optional, may be used to guide a catheter onto the ends of the multi-stage barb fitting 200.

The barbs 211, 212, 221, 222 may be used to connect catheters of different inner diameters as shown in FIGS. 1B and 1C. Because the outer-most pair of barbs 211, 212 have smaller diameters, when connecting a catheter with a larger inner diameter (e.g., the first catheter 10), such a larger inner diameter catheter may be slid past one of the outer-most barbs (e.g., the first-end first barb 211) and over one of the inner-most barbs (e.g., the first-end second barb 221). In contrast, when connecting a catheter with a smaller inner diameter (e.g., the second catheter 20), such a smaller inner diameter catheter may not slide as far onto the multi-stage barb fitting 200 as a catheter with a larger inner diameter. Thus, such a smaller inner diameter catheter (e.g., the second catheter 20) may be slid past one of the outer-most barbs (e.g., the second-end first barb 212), but may not be able to slide over the inner-most barb (e.g., the second-end second barb 222).

The inner landings 215, 216, 225, 226, which provide an offset between barbs 211, 212, 221, 222, may also encourage the catheters 10, 20 to better grip the respective barbs 211, 212, 221, 222 on which the catheters 10, 20 are mounted. A portion of the catheters 10, 20 are generally made to expand as they are slid over at least one of the barbs 211, 212, 221, 222, but will generally contract once that portion of the catheter 10, 20 is slid beyond the individual barb that made the portion expand.

The multi-stage barb fitting 200 may be formed from one or more of various materials, depending upon the types of catheters being joined and the application and/or location in which the catheters are being used. For example, the multi-stage barb fitting 200 may be machined from a titanium alloy such as Titanium 6A1-4V (i.e., unified numbering system (UNS) designation R56400).

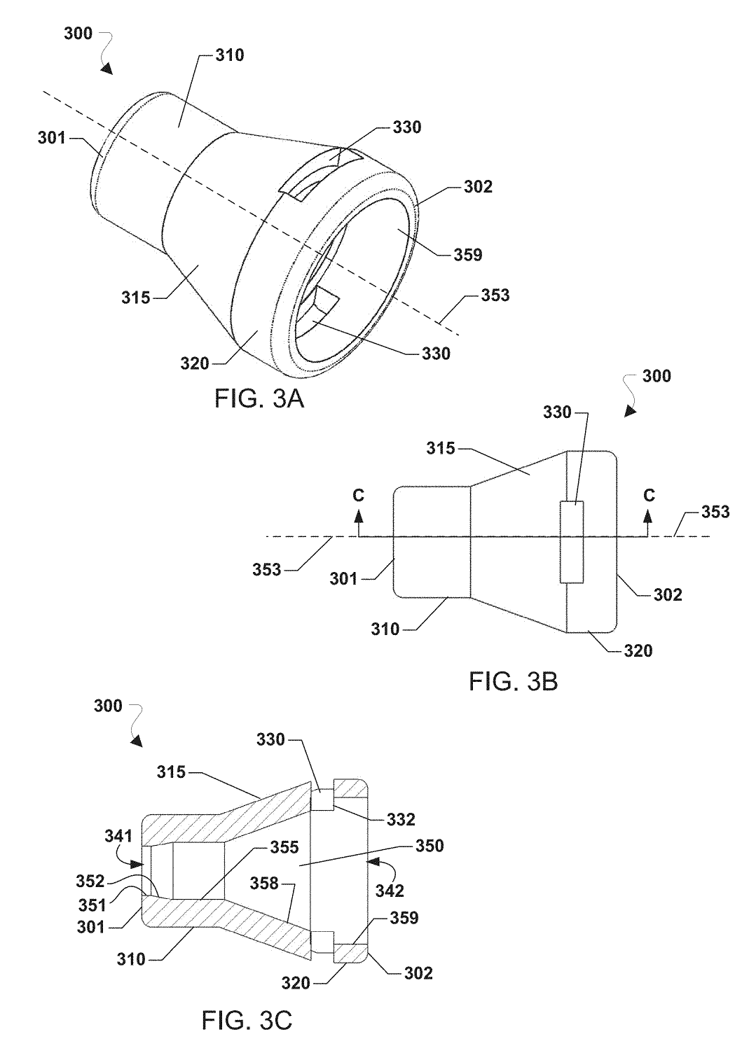

FIGS. 3A-3C illustrate a first locking member 300 in isolation in accordance with various embodiments. The first locking member 300 is configured to surround an end portion of a catheter (e.g., the first catheter 10) with a portion of one end of the multi-stage barb fitting (e.g., 200) inserted therein. In addition, the first locking member 300 is configured to interlock and mate together with the second locking member 400 and surround the multi-stage barb fitting joining the two catheters (e.g., 10, 20).

In various embodiments, the first locking member 300 may include outer walls forming a first inner cavity 350 between a first entry aperture 341 and a first egress aperture 342, opposite the first entry aperture 341. The first entry aperture 341 and the first egress aperture 342 may both be centered on a first longitudinal axis 353 of the first locking member 300. When the locking catheter splice assembly (e.g., 100) is fully assembled in accordance with various embodiments, the first longitudinal axis 353 may be coincident with the central longitudinal axis (e.g., 250) of the multi-stage barb fitting (e.g., 200). The outer walls may include a first-end first cylindrical portion 310 at a first end 301 of the first locking member 300. In addition, the outer walls may include a first-end second cylindrical portion 320 at a second end 302 of the first locking member 300. Both an outer and inner diameter of the first-end second cylindrical portion 320 may be larger than an outer and inner diameter, respectively of the first-end first cylindrical portion 310. In addition, the first locking member 300 may include a conical portion 315 connecting the first-end first cylindrical portion 310 to the first-end second cylindrical portion 320.

The first locking member 300 may include means for interlocking the first locking member 300 with the second locking member 400. For example, one or more latching slots 330 on the first locking member 300 may form the means for interlocking with locking tabs 430 of the second locking member 400 as described. The one or more latching slots 330 may each include a lateral aperture through the outer walls of the first-end second cylindrical portion 320. Alternatively, the one or more latching slots 330 need not pass entirely through the outer walls, but include an inner recess large enough to catch a latching tab (e.g., 430) on the second locking member. Since separating the first locking member 300 from the second locking member may be difficult if the one or more latching slots 330 do not pass entirely through the outer wall, such an embodiment may be well suited for a single-use or disposable application of the locking catheter splice assembly 100 in accordance with various embodiments.

In various embodiments, the first locking member 300 may further include inner engagement surfaces for radially compressing one of the catheters being joined (e.g., the first catheter 10) to the multi-stage barb fitting 200. In particular, the first locking member 300 may include a first-end first inner engagement surface 352 sized and positioned to trap one portion of the surrounded catheter (e.g., 10) against one of the barbs on the multi-stage barb fitting (e.g., 200), such as the first-end first barb (e.g., 211). The first-end first inner engagement surface 352 may be formed as a conical inner surface between a first-end smallest diameter portion 351 and a first-end intermediate diameter cylindrical portion 355. In order to accommodate larger diameter catheters, the first locking member 300 may also include a first-end second inner engagement surface 358 sized and positioned to trap another portion of the surrounded catheter (e.g., 10) against another barb on the multi-stage barb fitting (e.g., 200), such as the first-end second barb (e.g., 221). The first-end second inner engagement surface 358 may be formed as a conical inner surface between the first-end intermediate diameter cylindrical portion 355 and a first-end larger diameter cylindrical portion 359. The first-end larger diameter cylindrical portion 359 may be sized and positioned to coincide with a position, along the longitudinal axis of the multi-stage barb fitting, of the central fitting structure (e.g., 230) once the locking catheter splice assembly 100 is fully assembled.

The first locking member 300 may be formed from one or more of various materials, depending upon the types of catheters being joined and the application and/or location in which the catheters are being used. For example, the first locking member 300 may be formed, by molding, of an implantable grade thermoplastic polymer, such as some polysulfones (e.g., Eviva™ EV-500 by Solvay, Princeton, N.J.). Optionally, the thermoplastic polymer may be clear, semi-transparent, or at least translucent, to allow a clinician to see inside the locking catheter splice assembly 100 once fully assembled and allow the clinician to visually inspect the integrity of the catheter coupling during assembly.

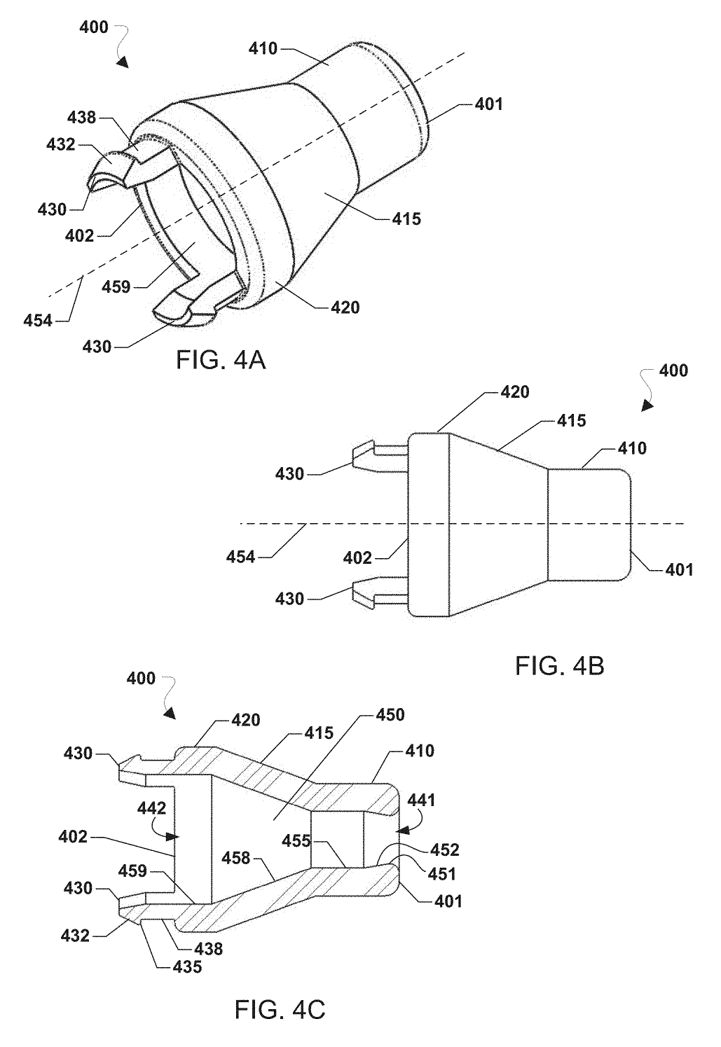

FIGS. 4A-4C illustrate a second locking member 400 in isolation in accordance with various embodiments. The second locking member 400 is configured to surround an end portion of another catheter (e.g., the second catheter 20) with a portion of the multi-stage barb fitting 200 inserted therein. In addition, the second locking member 400 is configured to interlock and mate together with the first locking member 300, while surrounding the multi-stage barb fitting joining the two catheters (e.g., 10, 20).

In various embodiments, the second locking member 400 may include outer walls forming a second inner cavity 450 between a second entry aperture 441 and a second egress aperture 442, opposite the second entry aperture 441. The second entry aperture 441 and the second egress aperture 442 may both be centered on a second longitudinal axis 454 of the second locking member 400. The outer walls may include a second-end first cylindrical portion 410 at a first end 401 of the second locking member 400. In addition, the outer walls may include a second-end second cylindrical portion 420 at a second end 402 of the second locking member 400. Both an outer and inner diameter of the second-end second cylindrical portion 420 may be larger than an outer and inner diameter, respectively of the second-end first cylindrical portion 410. In addition, the second locking member 400 may include a conical portion 415 connecting the second-end first cylindrical portion 410 to the second-end second cylindrical portion 420.

The second locking member 400 may include means for interlocking the second locking member 400 with the first locking member 300. For example, one or more latching tabs 430 on the second locking member 400 may form a means for interlocking with latching slots 330 of the first locking member 300 as described above. The one or more latching tabs 430 may each include a protruding arm extending away from the second end 402, in a direction parallel to or roughly parallel to the direction of the second longitudinal axis 454. When the locking catheter splice assembly 100 is fully assembled in accordance with various embodiments, the second longitudinal axis 454 may be coincident with the central longitudinal axis 250 of the multi-stage barb fitting 200.

Each of the one or more latching tabs 430 may include a base arm portion 438 extending from the second end 402 and a remote head 432, furthest from the second end 402. The base arm portion 438 may have an outer surface, positioned radially closer to the second longitudinal axis 454 than a catch peak 435 of the remote head 432 or an outer surface of the second-end second cylindrical portion 420. The remote head 432 may include an inclined surface facing away from the second end 402 and rises to the catch peak 435.

The included surface may be configured to temporarily deflect the remote head toward the second longitudinal axis 454 when the second locking member 400 is initially being coupled to the first locking member 300. As the one or more latching tabs 430 are pushed further inside the first-end larger diameter cylindrical portion 359 of the first locking member 300, the catch peak 435 on each of the latching tabs 430 will eventually reach a latching slot 330 of the first locking member. The latching tabs 430 may be resilient, such that once the first and second locking members are push far enough together, the catch peak 435 will snap into a latching slot and the respective latching tab 430 will bend back to its pre-deflected state to lock the first and second locking members together.

In various embodiments, the second locking member 400 may further include inner engagement surfaces for radially compressing the other one of the catheters being joined (e.g., the second catheter 20) to the multi-stage barb fitting 200. In particular, the second locking member 400 may include a second-end first inner engagement surface 452 sized and positioned to trap one portion of the surrounded catheter (e.g., 20) against one of the barbs on the multi-stage barb fitting 200, such as the second-end first barb 212. The second-end first inner engagement surface 452 may be formed as a conical inner surface between a second-end smallest diameter portion 451 and a second-end intermediate diameter cylindrical portion 455. In order to accommodate larger diameter catheters, the second locking member 400 may include a second-end second inner engagement surface 458 sized and positioned to trap another portion of the surrounded catheter (e.g., 20) against another barb on the multi-stage barb fitting 200, such as the second-end second barb 222. The second-end second inner engagement surface 458 may be formed as a conical inner surface between the second-end intermediate diameter cylindrical portion 455 and a second-end larger diameter cylindrical portion 459. The second-end larger diameter cylindrical portion 459 may be sized and positioned to coincide with a position, along the longitudinal axis of the multi-stage barb fitting, of the central fitting structure 230 once the locking catheter splice assembly 100 is fully assembled.

The second locking member 400, like the first locking member 300, may be formed from one or more of various materials, depending upon the types of catheters being joined and the application and/or location in which the catheters are being used. For example, the second locking member 400 may be formed by molding of an implantable grade thermoplastic polymer, such as some polysulfones (e.g., Eviva™ EV-500 by Solvay, Princeton, N.J.). Optionally, the thermoplastic polymer may be clear, semi-transparent, or at least translucent to enable a clinician to see inside the locking catheter splice assembly 100 once fully assembled and allow the clinician to visually inspect the integrity of the catheter coupling during assembly.

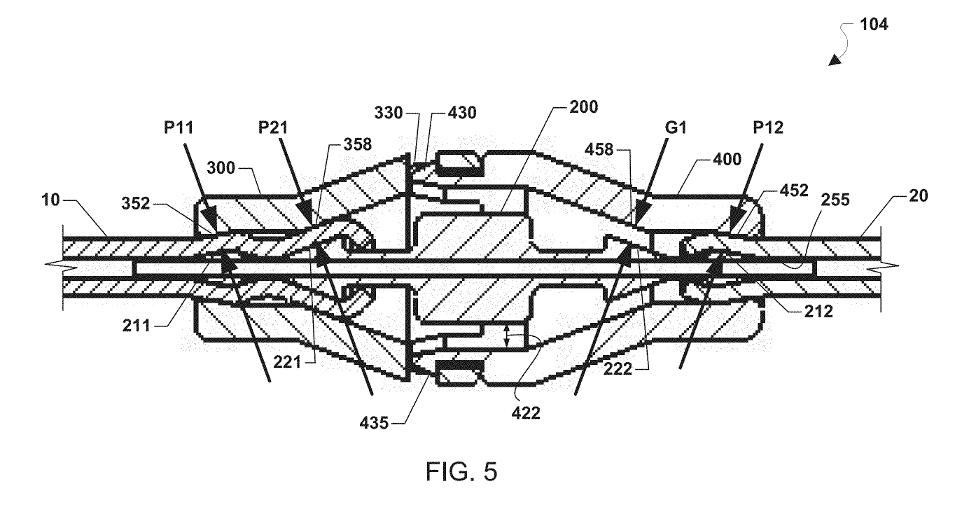

FIG. 5 illustrates further details of the locking catheter splice assembly 100 in a fully assembled state, with the first and second locking members 300, 400 interlocked together and covering the multi-stage barb fitting 200 that is connected to the first and second catheters 10, 20. In the fully assembled state, the latching tabs 430 of the second locking member 400 have been snapped into place and are seated at least partially in the latching slots 330 of the first locking member 300. Prior to the fully assembled state, when the first and second locking members 300, 400 are being pushed together, the latching tabs 430 may be deflected radially inwardly toward the central fitting structure 230 of the multi-stage barb fitting 200. In order to accommodate this deflection, an offset or gap 422 may be provided between an outer diameter of the central fitting structure 230 and an innermost radial position of an inner end of each latching tab 430.

Once the locking catheter splice assembly 100 is fully assembled and connecting the first and second catheters 10, 20, the inner lumen 255 of the multi-stage barb fitting 200 may channel any fluid from one of the catheters to the other. In addition, the first and second locking members 300, 400, together with the multi-stage barb fitting 200 are configured to join and hold the first and second catheters 10, 20 together. At least one of the various inner engagement surfaces (e.g., 352, 358, 452, 458) of each of the first and second locking members 300, 400 are configured to squeeze one or two portions of the first and second catheters 10, 20 into the barbs of the multi-stage barb fitting 200. Using the various inner engagement surfaces and the different barbs of the multi-stage barb fitting 200, the locking catheter splice assembly 100 can join two different catheters of the same or different inner diameter.

In the fully assembled state of the locking catheter splice assembly 100, the first-end first inner engagement surface 352 of the first locking member 300 may press and/or trap one portion P11 of the first catheter 10 against the first-end first barb 211 of the multi-stage barb fitting 200. In addition, if the first catheter 10 has a large enough inner diameter or is flexible enough, the multi-stage barb fitting 200 may be inserted far enough into the first catheter 10 that another portion P21 of the first catheter 10 covers the first-end second barb 221 of the multi-stage barb fitting 200. With the other portion P21 of the first catheter covering the first-end second barb 221, the first-end second inner engagement surface 358 of the first locking member 300 may press and/or trap the other portion P21 of the first catheter 10 against the first-end second barb 221 of the multi-stage barb fitting 200.

Also in the fully assembled state of the locking catheter splice assembly 100, the second-end first inner engagement surface 452 of the second locking member 400 may press and/or trap one portion P12 of the second catheter 20 against the second-end first barb 211 of the multi-stage barb fitting 200. In the illustrated example, the second catheter 20 has a large enough inner diameter or is flexible enough to cover the second-end second barb 222. In this case, the second-end second inner engagement surface 458 of the second locking member 400 may not press and/or trap any portion of the second catheter 20, thus leaving a gap G1 between the second-end second inner engagement surface 458 and the second-end second barb 222 of the multi-stage barb fitting 200.

A pressure force from the first and second catheters 10, 20 being compressed at the trapped portions P11, P21, P12 may tend to push the first and second locking members 300, 400 away from one another, which may be resisted by the catch peak 435 of the latching tab 430 seating in the latching slot 330. That pressure force may hold the catch peak 435 against one end of the latching slot 330 and prevent the first and second locking members 300, 400 from moving relative to one another and potentially separating.

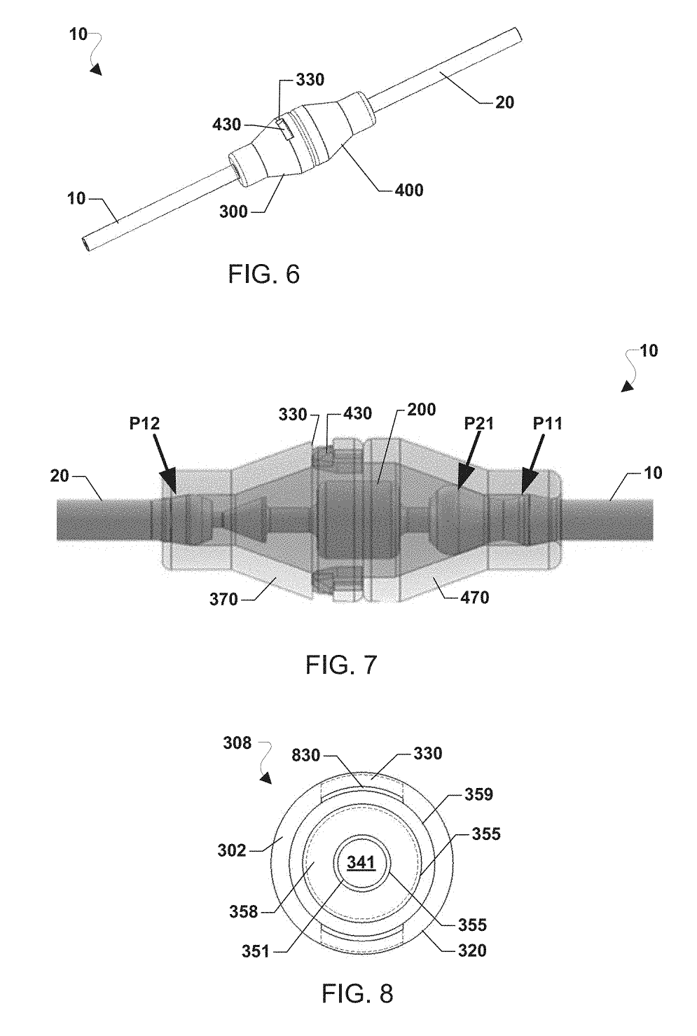

FIG. 6 illustrates a perspective view of the assembled locking catheter splice assembly 100 joining the first and second catheters 10, 20 in accordance with various embodiments. Once assembled, the locking catheter splice assembly 100 may not come apart easily. In some embodiments, a clinician may insert a tool into the latching slot 330 from the outside in order to press the latching tabs 430 inwardly, to release the first and second locking members 300, 400 from one another. In some embodiments, the first and second locking members 300, 400 may not be disconnected once the locking catheter splice assembly 100 has been assembled.

FIG. 7 illustrates an embodiment in which the first and second locking members 300, 400 are made of a clear, semi-transparent, or translucent material that allows a clinician to observe the connections of the first and second catheters 10, 20 within the locking catheter splice assembly 100. Looking through the walls 370, 470 of the first and second locking members 300, 400, the clinician may confirm that a sufficient amount of each of the first and second catheters 10, 20 is held by the multi-stage barb fitting 200 before finally pressing the locking members together. For example, each of the first and second catheters 10, 20 should have at least one trapped portion (e.g., P11, P21, P11). In addition, the clinician may confirm that each latching tab 430 is well seated in a latching slot 330 after assembly.

FIG. 8 illustrates an end view of an alternative first locking member 308 that includes key slots 830 for guiding latching tabs 430 into the latching slot 330. The key slots 830 may be formed as depressions into the first-end larger diameter cylindrical portion 359 of the alternative first locking member 308. Otherwise, various other elements of the alternative first locking member 308 may be the same or similar to that described with reference to FIGS. 1-7 for the first locking member 300. For example, the alternative first locking member 308 may include the second end 302 with the first-end second cylindrical portion 320, the first entry aperture 341 (e.g., at the first end 301), the first-end smallest diameter portion 351, the first-end intermediate diameter cylindrical portion 355, the first-end second inner engagement surface 358, the first-end intermediate diameter cylindrical portion 355, and the like.

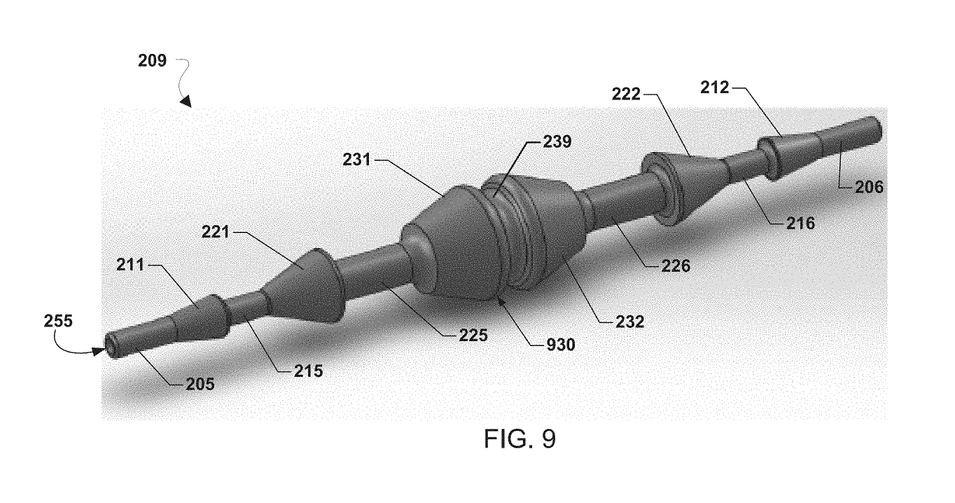

FIG. 9 illustrates a perspective view of an alternative multi-stage barb fitting 209 in accordance with various embodiments. The alternative multi-stage barb fitting 209 may include a symmetrically tapered central fitting structure 930 having first and second oppositely facing barb-like structures 231, 232 and a central recess 239. The first and second oppositely facing barb-like structures 231, 232 may form a third larger sized barb for accommodating even a greater range of catheter diameters. The central recess 239 may accommodate the end of at least one of the catheters (e.g., 15, 25). Various other elements of the alternative multi-stage barb fitting 209 may be the same or similar to that described herein for the multi-stage barb fitting 200 described herein. For example, the alternative multi-stage barb fitting 209 may include the outermost landings 205, 206, the outer-most pair of barbs 211, 212, intermediate landings 215, 216, the inner-most pair of barbs 221, 222, the inner-most landings 225, 226, and the inner lumen 255.

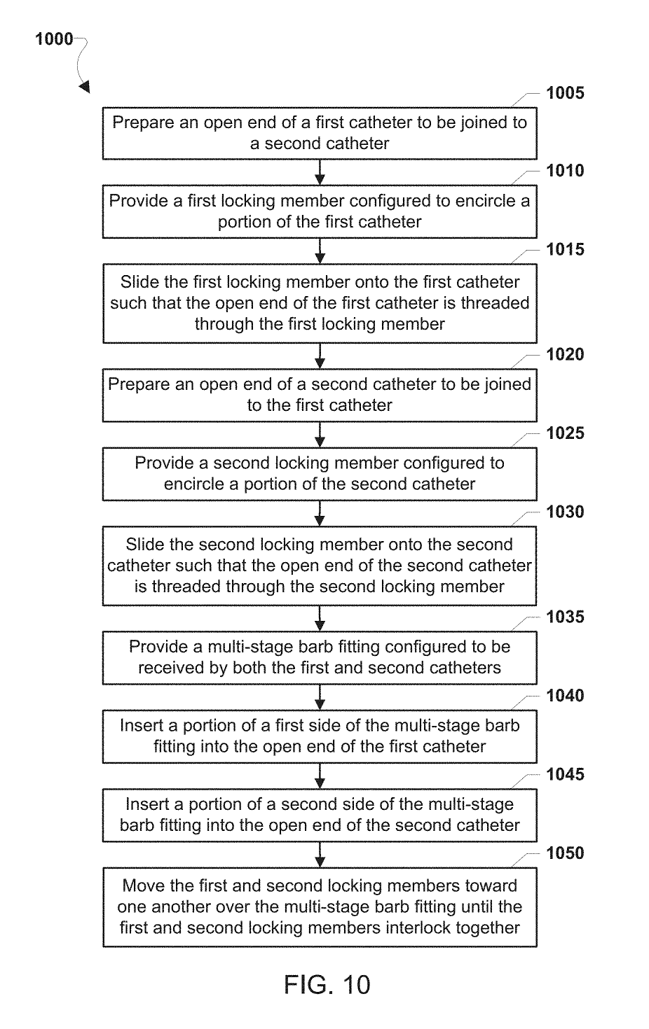

FIG. 10 is a process flow diagram of an embodiment method 1000 for joining a first catheter and a second catheter in accordance with various embodiments. The methods and means of joining two catheters in accordance with various embodiments, may not only provide a solid and reliable way of joining two catheters, but may also simplifying splicing the two catheters in a medical procedure. In various surgical settings, such as when joining catheters connecting an implantable infusion pump, the entire locking catheter splice assembly may comprise tiny parts that are difficult to manipulate by a clinician. By first mounting the first and second locking members (e.g., 300, 400) on the respective first and second catheters (e.g., 10, 20), the first and second locking members are not at risk of being dropped. Once mounted in this way, the first and second locking members are ready to be slid into their final position after joining the multi-stage barb fitting (e.g., 200) to the first and second catheters. Once the multi-stage barb fitting is secured to the first and second catheters, the clinician may slide the first and second locking members toward one another until they mate over the multi-stage barb fitting, and the latching elements engage. Sliding the first and second locking members in this way may be guided by the first and second catheters on which the first and second locking members are mounted. Mating the first and second locking members in this way may secure each section of catheter by means of radially compression of each catheter between the multi-stage barb fitting and the first and second locking members.

Thus, a clinician may perform the method 1000 for joining two catheters using a locking catheter splice assembly (e.g., 100) in accordance with various embodiments. In block 1005, the clinician may prepare an open end (e.g., 15) of a first catheter to be joined to the second catheter. The clinician may inspect the open end of the first catheter to ensure it is clean, clear, and otherwise ready to receive the multi-stage barb fitting therein. The clinician may need to cut-off a portion of the first catheter to ensure the open end has a smooth and clean cut and that the first catheter is of the appropriate length.

In block 1010, the clinician may provide or obtain a first locking member (e.g., 300, 308) configured to surround a portion of the first catheter, and slide the first locking member onto the first catheter in block 1015, such as illustrated in FIG. 1A.

In block 1020, the clinician may prepare an open end of a second catheter to be joined to the first catheter. The clinician may inspect the open end of the second catheter to ensure it is clean, clear, and otherwise ready to receive the multi-stage barb fitting therein. The clinician may need to cut-off a portion of the second catheter to ensure the open end has a smooth and clean cut and that the second catheter is of the appropriate length.

In block 1025, the clinician may provide or obtain a second locking member (e.g., 400) configured to surround a portion of the second catheter, and slide the second locking member onto the second catheter in block 1030, such as illustrated in FIG. 1A.

In block 1035, the clinician may provide or obtain a multi-stage barb fitting 200, 209 configured as described herein.

In block 1040, the clinician may insert a portion of a first end of the multi-stage barb fitting into the open end of the first catheter, such as illustrated in FIG. 1B.

In block 1045, the clinician may insert a portion of a second end of the multi-stage barb fitting into the open end of the second catheter, such as illustrated in FIG. 1B.

In block 1050, the clinician may move the first and second locking members toward one another over the multi-stage barb fitting until the first and second locking members interlock together, such as illustrated in FIGS. 1C and 5-7. Before pressing the first and second locking members together sufficient to engage the interlocks, the clinician may look through the first and second locking members in some embodiments to confirm that the catheters are properly engaged on the multi-stage barb fitting and that the locking members are positioned to engage a bulge portion of the catheters. In some embodiments, the clinician may look through the first and second locking members after they have been pressed together to confirm that the latches have properly engaged.

While several embodiments of a locking catheter splice assembly are described above in connection with a method and system of joining two catheters, the embodiments may be applicable to a variety of catheter or tube splicing systems, and in particular to fluid flow systems that are implantable in a patient or otherwise not easily accessible because the various embodiments may be used to join two tubular elements, and particularly two catheters.

The foregoing method descriptions and the process flow diagram are provided merely as illustrative examples and are not intended to require or imply that the blocks of the various aspects must be performed in the order presented. As will be appreciated by one of skill in the art the order of blocks in the foregoing aspects may be performed in any order. Words such as “thereafter,” “then,” “next,” etc. are not intended to limit the order of the blocks; these words are simply used to guide the reader through the description of the methods. Further, references to elements or parts moving “left,” “right,” “up,” “down,” “upwardly,” “downwardly,” “inwardly,” and/or “outwardly” are merely for relating movements of the parts in the orientation illustrated in the figures, and are not intended to limit the scope of the claims regarding a particular orientation of device or diaphragm with respect to the Earth. Further, any reference to claim elements in the singular, for example, using the articles “a,” “an” or “the” is not to be construed as limiting any element to the singular.

The previous description of the disclosed embodiments is provided to enable any person skilled in the art to make or use the present claims. Various modifications to these embodiments will be readily apparent to those skilled in the art, and the generic principles defined herein may be applied to other embodiments without departing from the scope of the claims. Thus, the claims are not intended to be limited to the embodiments shown herein but are to be accorded the widest scope consistent with the principles and novel features disclosed herein.

What is claimed is:

1. A locking catheter splice assembly for joining a first catheter and a second catheter, comprising:
a multi-stage barb fitting having a first end including a first set of barbs and a second end including a second set of barbs, wherein the first end is configured to be inserted into the first catheter and the second end is configured to be inserted into the second catheter;
a first locking member configured to be slipped over the first catheter and to surround a portion of the first end of the multi-stage barb fitting inserted into the first catheter; and
a second locking member configured to be slipped over the second catheter and to surround the portion of the second end of the multi-stage barb fitting inserted into the second catheter,
wherein the first and second locking members are configured to interlock with one another when pressed together.

2. The locking catheter splice assembly of claim 1, wherein the first locking member is configured to compress an outer diameter of a portion of the first catheter against at least one of the first set of barbs when the first and second locking members are interlocked with one another.

3. The locking catheter splice assembly of claim 1, wherein the first locking member includes an inner cavity having a first catheter trapping surface separate from a second catheter trapping surface, wherein the first and second catheter trapping surfaces are axially aligned with a first barb and a second barb when the first locking member and the second locking member are interlocked with one another.

4. The locking catheter splice assembly of claim 1, wherein each of the first and second sets of barbs include a first barb and a second barb, wherein the first barb has a smaller outermost diameter than the second barb.

5. The locking catheter splice assembly of claim 1, wherein the multi-stage barb fitting is symmetrical about a longitudinal axis.

6. The locking catheter splice assembly of claim 1, wherein one of the first and second locking members includes a latching tab configured to be received in a latching slot included in the other of the first and second locking members for interlocking the first and second locking members.

7. The locking catheter splice assembly of claim 6, wherein the latching slot extends radially through the other of the first and second locking members that includes the latching slot.

8. The locking catheter splice assembly of claim 6, wherein the other of the first and second locking members that includes the latching slot further includes a guide slot configured to guide movement of the first and second locking members toward one another along a longitudinal axis of the multi-stage barb fitting.

9. The locking catheter splice assembly of claim 1, wherein the multi-stage barb fitting includes a central portion positioned between the first and second ends that is wider than the first and second sets of barbs.

10. The locking catheter splice assembly of claim 9, wherein the central portion includes a gripping surface configured to facilitate gripping of the central portion during surgery.

11. The locking catheter splice assembly of claim 9, wherein a gap is formed between a largest outer diameter of the central portion and a largest inner diameter of at least one of the first and second locking members when the first and second locking members are mated together to couple the first and second catheters using the multi-stage barb fitting.

12. A method for joining a first catheter and a second catheter, comprising:
slipping a first locking member over the first catheter;
slipping a second locking member over the second catheter;
inserting a multi-stage barb fitting into open ends of both the first and second catheters, wherein a first end of the multi-stage barb fitting includes a first set of barbs and a second end of the multi-stage barb fitting includes a second set of barbs; and
pressing the first and second locking members together over the multi-stage barb fitting until the first and second locking members interlock.
